(19) United States
(12) Patent Application Publication
(10) Pub. No.: US 2012/0116368 A1
(43) Pub. Date: May 10, 2012

(54) SURGICAL INSTRUMENT WITH ADD-ON POWER ADAPTER FOR ACCESSORY

(76) Inventor: Frank J. Viola, Sandy Hook, CT (US)

(21) Appl. No.: 13/281,569

(22) Filed: Oct. 26, 2011

Related U.S. Application Data
(60) Provisional application No. 61/412,115, filed on Nov. 10, 2010.

(51) Int. Cl. A61B 17/00

(52) U.S. Cl. 606/1

(57) ABSTRACT

A surgical instrument system includes a surgical instrument, an accessory, and an adapter. The surgical instrument includes a housing, a shaft extending from the housing, and a tool assembly operably coupled to the shaft. The accessory is operably couplable to the surgical instrument. The adapter is operably coupled to the surgical instrument and includes an energy storage device to provide power to the accessory when the accessory is operably coupled to the surgical instrument.

This application claims priority from provisional application Ser. No. 61/412,115, filed Nov. 10, 2010, the entire contents of which are incorporated herein by reference.

### BACKGROUND

1. Technical Field

This application relates to surgical instruments and more particularly, to an adapter for surgical instruments that provides power to accessories for use with surgical instruments.

2. Background of Related Art

A typical surgery employs a plurality of different surgical instruments and accessory devices for use with the various surgical instruments. When attaching accessory devices, e.g. illumination devices or cameras, there is often a need to satisfy the energy needs of the accessory device. While self contained energy sources like batteries are often utilized, they take up valuable space in the accessory device, thereby increasing the size of the accessory which is disadvantageous in minimally invasive surgery. Also, such energy sources often have limited energy storage capacity. As such, removal or repositioning of the accessory may be necessary to change a battery or other energy storage device, which, if required during surgery or other medical procedure, can increase the time and complexity of the surgery and reduce efficiency.

### SUMMARY

Accordingly, the present disclosure in one aspect is directed to a surgical instrument system that includes a surgical instrument, an accessory, and an adapter. The surgical instrument includes a housing, a shaft extending from the housing, and a tool assembly operably coupled to the shaft. The accessory is operably couplable to the surgical instrument. The adapter is operably coupled to the surgical instrument and includes an energy storage device to provide power to the accessory when the accessory is operably coupled to the surgical instrument.

The energy storage device may be a battery. The accessory may include one or more of a camera and an illumination device.

In one embodiment, the surgical instrument includes a base portion disposed adjacent one or more of the shaft and the housing. The base portion can be configured and dimensioned to engage the adapter such that the adapter transmits power to the base portion and the base portion transmits power to the accessory. The base portion may include one or more contacts.

The adapter may include one or more contacts. The one or more contacts of the adapter and the one or more contacts of the base portion can be configured and dimensioned to engage one another to transmit the power from the adapter to the base portion. The adapter can define an energy storage housing for positioning a battery therein. The adapter can be slidably coupled onto the shaft. In some embodiments, the adapter is operably coupled to the surgical instrument via a slip ring in order to transmit the power from the adapter.

The accessory can include at least one contact to engage a contact of the instrument. The contact for the accessory can be distal of the contact for the adapter.

In another aspect, the present disclosure provides a surgical instrument system for powering an accessory comprising a shaft supporting at least one contact and an adapter operably coupled to the shaft. The adapter includes an energy storage device to provide power to the accessory when the accessory is operably coupled to the surgical instrument in electrical connection with the at least one contact.

In some embodiments, the surgical instrument includes a base portion disposed adjacent to at least one of the shaft and the housing, and the adapter engages the base portion of the surgical instrument such that the adapter transmits power to the base portion and the base portion transmits power to the accessory.

In some embodiments, the adapter includes at least one contact and the surgical instrument includes at least one contact, wherein the contacts are configured and dimensioned to engage one another to transmit the power from the adapter to the surgical instrument for transmission to the accessory.

### BRIEF DESCRIPTION OF THE DRAWINGS

Various embodiments of the present disclosure are described herein with reference to the drawings wherein:

FIG. 1 is a perspective view of one embodiment of a surgical instrument system in accordance with the present disclosure;

FIG. 1A is a perspective view of the accessory and distal portion of the instrument of FIG. 1;

FIG. 2 is a partial perspective view, with parts separated, illustrating the positioning of one embodiment of an adapter on the surgical instrument system of FIG. 1;

FIG. 3 is a bottom perspective view of the adapter illustrated in FIG. 2;

FIG. 4 is a partial perspective view, with parts separated, illustrating the positioning of another embodiment of an adapter on another embodiment of a surgical instrument;

FIG. 5 is a bottom perspective view of the adapter illustrated in FIG. 4; and

FIG. 6 is a perspective of another instrument of the present disclosure.

### DETAILED DESCRIPTION

Embodiments of the presently disclosed surgical instrument are described in detail with reference to the drawings, wherein like reference numerals designate similar or identical elements in each of the several views. In the drawings and the description that follows, the term “proximal” refers to the end of the surgical instrument that is closer to the user, whereas the term “distal” refers to the end of the surgical instrument that is further from the user.

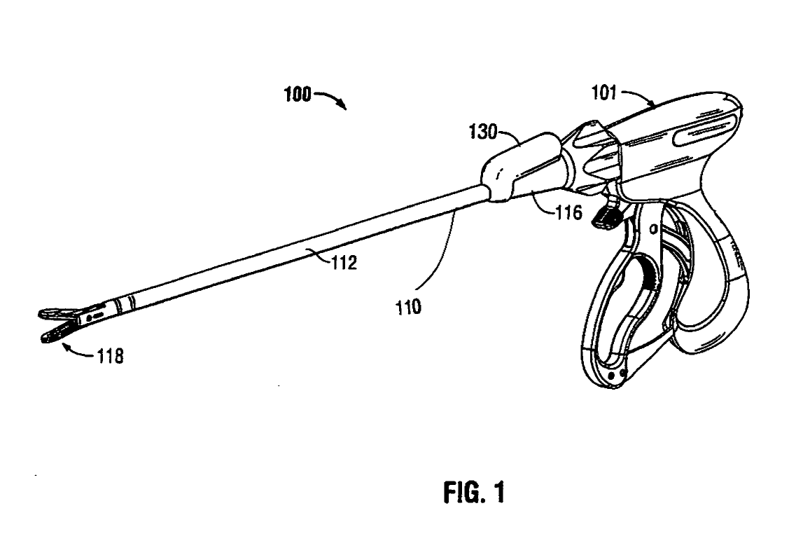

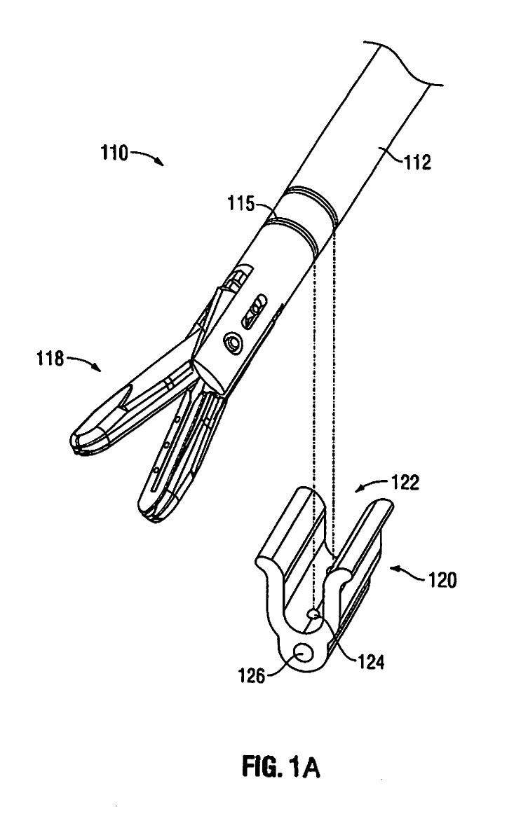

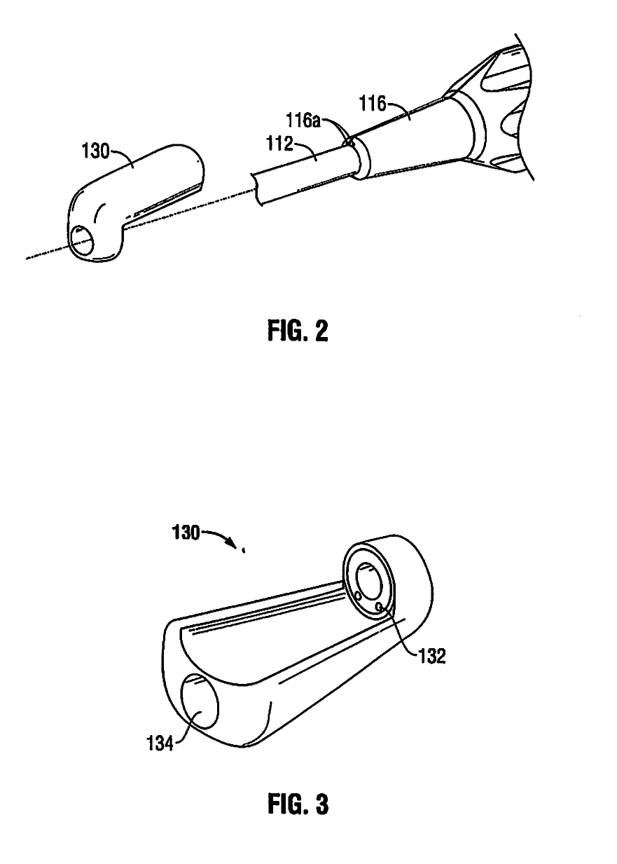

Referring now to FIGS. 1 and 2, one embodiment of a surgical instrument system 100 is illustrated. The surgical instrument system 100 includes a surgical instrument 101, an adapter 130, and an accessory 120. The instrument illustrated is an endoscopic grasper, by way of example, it being understood that other instruments can also be utilized such as endoscopic scissors, clip appliers, surgical staplers, etc. The surgical instrument 101 has a housing 110, an elongated shaft 112, a base portion 116, and a tool assembly 118. The shaft 112 extends from the housing 110 and base portion 116. The tool assembly 118 is disposed on the distal end portion of the shaft 112. The base portion 116 is disposed at a proximal end portion of shaft 112.

The surgical instrument 110 includes one or more contacts 115. In the illustrated embodiment, two ring contacts 115 are shown by way of example. The contacts 115 may be positioned on the shaft 112. The contacts 115 may include contact buttons, slip rings, or any other suitable connection. The contacts 115 are electrically coupled to the adapter 130, which contains an energy storage device, e.g., via one or more wires extending therebetween (through the shaft 112) and/or a wireless connection such that the energy storage device transmits power to the contacts 115. The contacts 115 are engaged by the contacts 124 of the accessory 120. In this manner, the power is transmitted to the accessory 120 from the adapter 130 through the contacts 115, 124.

Referring now to FIG. 1A, the accessory 120 is mountable, and preferably removably mountable, to the surgical instrument 110. The accessory 120 defines a channel 122 therethrough and includes one or more contacts 124. The channel 122 is configured and dimensioned to accommodate at least a portion of the shaft 112 when the accessory 120 is selectively operably coupled to the surgical instrument 110. Thus, the accessory 120 can frictionally engage the shaft 112. Alternatively, the accessory can be attached to the shaft by a snap fit, interlocking structure or by other methods. The contacts 124 may be positioned within the channel 122 such that when the accessory 120 is positioned on the shaft 112 adjacent contacts 115, two or more contacts (i.e., at least one contact 115 and at least one contact 124) are engaged so that the accessory 120 is electrically coupled to the energy device of adapter 130. In particular, the contacts 124 may be operably coupled to the energy device via any suitable electrical or electromechanical features. For example, these features, which may include wired or wireless connections, may have inductive components, capacitive components, resistive components, switching components, etc. that facilitate the connection between the accessory 120 and the surgical instrument 110 so that the accessory 120 can be powered by the energy device of adapter 130. Furthermore, the contacts 124 may include contact buttons, slip rings, or any other suitable connection components.

As illustrated in FIG. 2, the adapter 130 may be selectively operably coupled to the shaft 112 and/or the base portion 116 of the surgical instrument 100. In alternate embodiments, the adapter 130 is configured and dimensioned to couple to the housing of the surgical instrument. The adapter in some embodiments can be removably coupled to the instrument. As discussed above, the adapter 130 contains an energy storage device (e.g., a battery or any other suitable self-contained electrical energy source) that provides power/energy to the accessory 120 (e.g., via a wired and/or wireless connection).

It should be appreciated that other energy sources for powering the accessory 120 may also be stored in the adapter 130. The accessory 120 may be operably coupled to one or both of the adapter 130 and the surgical instrument 100 (e.g., the shaft 112, the housing 110, and/or the base portion 116). The accessory 120 may include one or more powered devices including cameras, illumination devices, or any other suitable powered device that could assist the clinician in performing a medical procedure.

The base portion 116 is configured and dimensioned to receive and engage the adapter 130 such that the adapter 130 transmits power to the base portion 116 for providing power to the accessory 120. The base portion 116 is operably coupled to the accessory 120. In this manner, the base portion 116 transmits power from the energy storage device, through the adapter 130, and to the accessory 120.

As illustrated in FIGS. 2 and 3, the base portion 116 includes one or more contact buttons 116a and the adapter 130 includes one or more contact buttons 132. The one or more contact buttons 132 of the adapter 130 and the one or more contact buttons 116a of the base portion 116 are configured and dimensioned to engage one another to transmit the power from the energy storage device to the accessory 120. The adapter 130 slidably couples onto the shaft 112 such that the contact buttons 132, 116a engage one another. The adapter 130 can attach to the shaft 112 by a frictional fit, snap fit or by other methods. The adapter 130 defines an energy storage housing 134 for positioning the energy storage device therein. The energy storage housing 134 facilitates the transmission of power from the energy storage device through the contact buttons 116a, 132 by enabling the energy storage device to be positioned in electrical communication with the contact buttons 116a, 132. In this respect, the energy storage device may be in direct contact with one or more of the contact buttons 116a, 132 when positioned in the energy storage housing 134. Note in the illustrated embodiments, contact buttons 116a are positioned proximally of the more distal contacts 115.

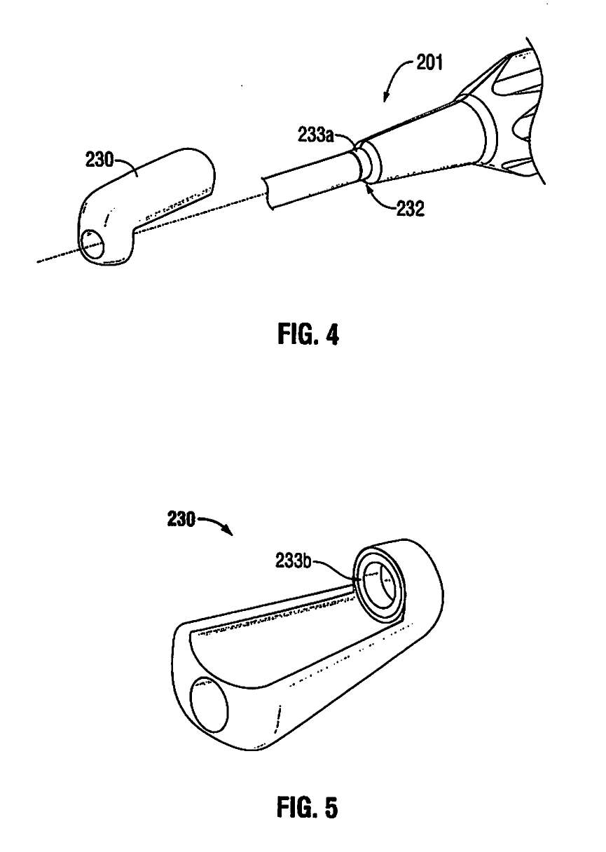

Referring now to FIGS. 4 and 5, another embodiment of a surgical instrument 201 and adapter 230 are illustrated. In this embodiment, the adapter 230 is operably coupled to the surgical instrument 201 via a slip ring arrangement 232 in order to transmit power from the energy storage device to the accessory 120. In this manner, a ring 233a may be positioned on the shaft 112 that engages an inner annular surface 233b of the adapter 230 to facilitate the transmission of power therethrough to provide power to the accessory 120.

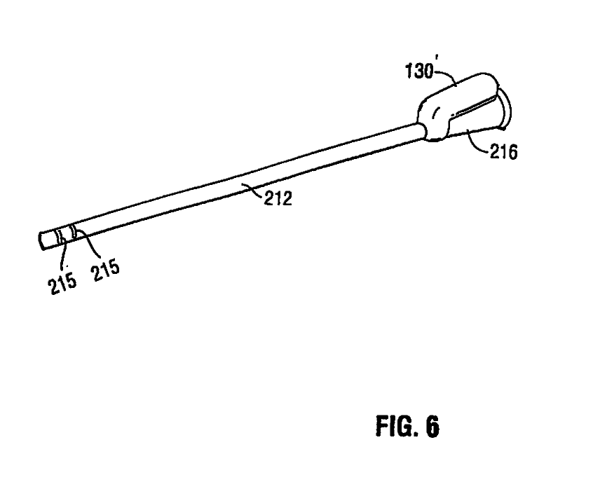

In the embodiment of FIG. 6, adapter 130′ can be attached to rod 212 adjacent base portion 216. An accessory, such as accessory 120 of FIG. 2, can be mounted to engage the contacts 215 positioned on rod 212. Contacts 215 can be wired to the energy storage device in adapter 130′ in the same manner as in FIG. 1 to supply energy to the accessory 120 when mounted to the rod 212. In this manner a simple device for inserting an accessory, e.g. a camera, into a body cavity is provided.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument system comprising:
a surgical instrument including
a housing;
a shaft extending from the housing; and
a tool assembly operably coupled to the shaft;
an accessory operably couplable to the surgical instrument; and
an adapter operably coupled to the surgical instrument, the adapter including an energy storage device to provide power to the accessory when the accessory is operably coupled to the surgical instrument.

2. The surgical instrument system according to claim 1, wherein the surgical instrument includes a base portion disposed adjacent to at least one of the shaft and the housing, the adapter engaging the base portion when coupled to the surgical instrument such that the adapter transmits power to the base portion, and the base portion transmits power to the accessory.

3. The surgical instrument system according to claim 1, wherein the adapter includes at least one contact and the surgical instrument includes at least one contact, wherein the at least one contact of the adapter and the at least one contact of the surgical instrument are configured and dimensioned to engage one another to transmit the power from the adapter to the surgical instrument for transmission to the accessory.

4. The surgical instrument system according to claim 1, wherein the energy storage device is a battery.

5. The surgical instrument system according to claim 4, wherein the adapter defines an energy storage housing for positioning the battery therein.

6. The surgical instrument system according to claim 1, wherein the adapter is slidably coupled to the shaft.

7. The surgical instrument system according to claim 1, wherein the accessory is at least one of a camera and an illumination device.

8. The surgical instrument system according to claim 1, wherein the adapter is operably coupled to the surgical instrument via a slip ring in order to transmit the power from the adapter.

9. The surgical instrument system according to claim 1, wherein the accessory frictionally engages the shaft.

10. The surgical instrument system according to claim 3, wherein the accessory includes at least one contact to engage a distal contact of the surgical instrument.

11. The surgical instrument system according to claim 10, wherein the distal contact is positioned on the shaft.

12. A surgical instrument system for powering an accessory comprising a shaft supporting at least one contact and an adapter operably coupled to the shaft, the adapter including an energy storage device to provide power to the accessory when the accessory is operably coupled to the surgical instrument in electrical connection with the at least one contact.

13. The surgical instrument system according to claim 12, wherein the surgical instrument includes a base portion disposed adjacent to at least one of the shaft, the adapter engaging the base portion of the surgical instrument, such that the adapter transmits power to the base portion, and the base portion transmits power to the accessory.

14. The surgical instrument system according to claim 12, wherein the adapter includes at least one contact and the surgical instrument includes at least one contact, wherein the at least one contact of the adapter and the at least one contact of the surgical instrument are configured and dimensioned to engage one another to transmit the power from the adapter to the surgical instrument for transmissions to the accessory.

15. The surgical instrument system according to claim 12, wherein the energy storage device is a battery.

16. The surgical instrument system according to claim 12, wherein the adapter is slidably coupled to the shaft.

17. The surgical instrument system according to claim 12, wherein the adapter is operably coupled to the surgical instrument via a slip ring in order to transmit the power from the adapter.

18. The surgical instrument system according to claim 12, wherein the instrument includes a distally positioned contact to engage a contact of the accessory.
